(19) United States
(12) Patent Application Publication
(10) Pub. No.: US 2017/0196469 A1
(43) Pub. Date: Jul. 13, 2017

(54) OPTICAL BLOOD PRESSURE DETECTION DEVICE AND OPERATING METHOD THEREOF

(71) Applicant: PIXART IMAGING INC., Hsin-Chu County (TW)

(72) Inventors: Wei-Ru HAN, Hsin-Chu County (TW); Ren-Hau GU, Hsin-Chu County (TW); Chih-Yuan CHUANG, Hsin-Chu County (TW)

(21) Appl. No.: 15/350,619

(22) Filed: Nov. 14, 2016

(30) Foreign Application Priority Data
Jan. 12, 2016 (TW) 105100804

(51) Int. Cl. A61B 5/021; A61B 5/08; A61B 5/00

(52) U.S. Cl. CPC A61B 5/02125; A61B 5/0082; A61B 5/0816; A61B 5/7257; A61B 5/6831

(57) ABSTRACT

A blood pressure detection method includes the steps of: acquiring a PPG signal from a skin surface using a light sensing element; calculating a blood pressure corresponding to each pulse duration according to at least one pressure estimation model and a time difference between two feature points within one pulse duration; calculating a breathing period; averaging a plurality of blood pressures within the breathing period to generate an average blood pressure; and showing the average blood pressure with a display device.

### CROSS REFERENCE TO RELATED APPLICATION

This application claims the priority benefit of Taiwan Patent Application Serial Number 105100804, filed on Jan. 12, 2016, the full disclosure of which is incorporated herein by reference.

### BACKGROUND

1. Field of the Disclosure

This disclosure generally relates to a blood pressure detection device and, more particularly, to an optical blood pressure detection device based on photoplethysmography (PPG) signals and an operating method thereof

2. Description of the Related Art

Conventionally, to obtain more reliable detected values using a hemadynamometer, it is necessary to repeatedly measure blood pressures for a longer time by the hemadynamometer. In addition, the conventional hemadynamometer can only perform the passive measurement but is unable to perform so-called active measurement continuously. Accordingly, detected values of the conventional hemadynamometer are blood pressures only for particular conditions (e.g., the emotion and movement of a user having no significant change for a period of time), but can not truly reflect all conditions, e.g., sleep blood pressures.

Accordingly, it is necessary to provide a blood pressure detection device capable of continuously monitoring blood pressures.

### SUMMARY

The present disclosure provides an optical blood pressure detection device capable of acquiring blood pressures and respiration rates of a user using a photoplethysmography (PPG) signal and an operating method thereof

The present disclosure provides an optical blood pressure detection device and an operating method that average a plurality of blood pressures using a respiration cycle to obtain a more stable average blood pressure.

The present disclosure provides a blood pressure detection device including a light source, a light sensor and a processor. The light source is configured to illuminate a skin surface to allow light to pass through skin tissues under the skin surface. The light sensor is configured to detect ejected light from the skin tissues to generate a photoplethysmography (PPG) signal. The processor is configured to calculate at least one blood pressure corresponding to each pulse duration according to at least one blood pressure estimation model and a time difference between two feature points within one pulse duration of the PPG signal, calculate a respiration cycle, and average a plurality of blood pressures within the respiration cycle to generate an average blood pressure, wherein the at least one blood pressure estimation model includes a polynomial using the time difference between the two feature points within one pulse duration as a variable.

The present disclosure further provides a blood pressure detection device including a light source, a light sensor, a memory and a processor. The light source is configured to illuminate a skin surface to allow light to pass through skin tissues under the skin surface. The light sensor is configured to detect ejected light from the skin tissues to generate a photoplethysmography (PPG) signal. The memory is configured to store at least one calibration value, wherein the calibration value is a difference value between a measured blood pressure of a hemadynamometer and an estimated blood pressure. The processor is configured to calculate a blood pressure corresponding to each pulse duration according to at least one blood pressure estimation model and a time difference between two feature points within one pulse duration of the PPG signal, calculate a respiration cycle, average a plurality of blood pressures within the respiration cycle to generate an average blood pressure, and calibrate the average blood pressure with the calibration value, wherein the at least one blood pressure estimation model includes a polynomial using the time difference between the two feature points within one pulse duration as a variable.

The present disclosure further provides an operating method of a blood pressure detection device including the steps of: obtaining, by a light sensor, a photoplethysmography (PPG) signal from a skin surface; calculating, by a processor, a blood pressure corresponding to each pulse duration according to at least one blood pressure estimation model and a time difference between two feature points within one pulse duration of the PPG signal, wherein the at least one blood pressure estimation model includes a polynomial using the time difference between the two feature points within one pulse duration as a variable; calculating, by the processor, a respiration cycle; and averaging, by the processor, a plurality of blood pressures within the respiration cycle to generate an average blood pressure.

### BRIEF DESCRIPTION OF THE DRAWINGS

Other objects, advantages, and novel features of the present disclosure will become more apparent from the following detailed description when taken in conjunction with the accompanying drawings.

FIG. 1 is a schematic diagram of a photoplethysmography (PPG) signal detected by a blood pressure detection device according to one embodiment of the present disclosure.

FIG. 2 is a schematic diagram of blood pressures obtained by a blood pressure estimation model according to one embodiment of the present disclosure.

FIGS. 3A and 3B are usage states of a blood pressure detection device according to some embodiments of the present disclosure.

FIG. 4 is a schematic block diagram of a blood pressure detection device according to one embodiment of the present disclosure.

FIG. 5 is a schematic diagram of average blood pressures according to one embodiment of the present disclosure.

FIG. 6 is a flow chart of an operating method of a blood pressure detection device according to one embodiment of the present disclosure.

### DETAILED DESCRIPTION OF THE EMBODIMENT

It should be noted that, wherever possible, the same reference numbers will be used throughout the drawings to refer to the same or like parts.

A photoplethysmography (PPG) signal is consisted of two parts. When a systole occurs, the pressure and the blood volume in blood vessels of the whole body have a continuous variation. When a diastole occurs, said pressure decreases correspondingly, and the blood pumped-out in a previous systole heats the heart valve to cause so-called inflection.

Therefore, a complete PPG waveform includes a mixed effect of systole and the pressure from blood vessel walls. The PPG signal is obtainable by detecting a volume variation of blood vessels through optical measurements.

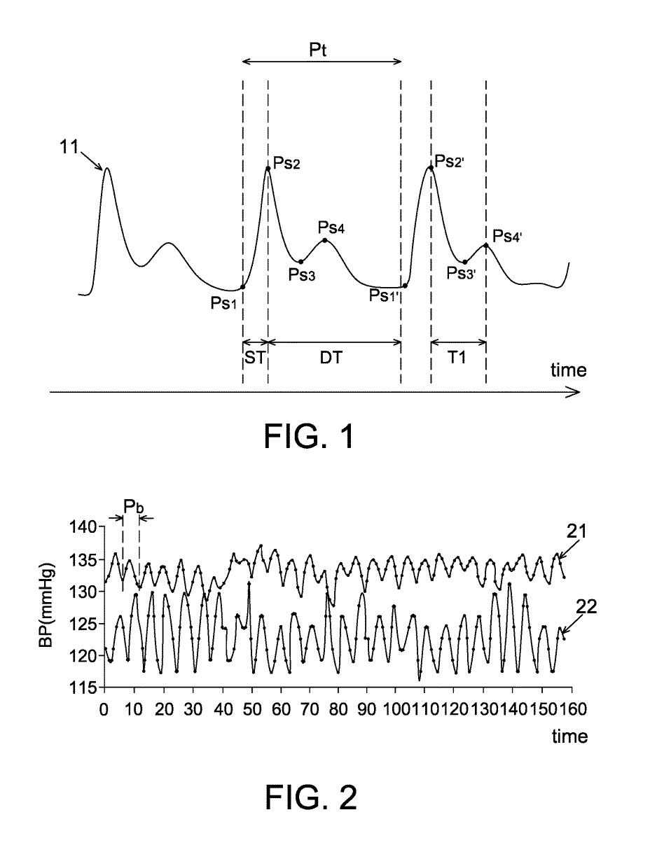

To obtain breathing signals and blood pressure signals of a user from a PPG signal, it is necessary to acquire the PPG signal at first. The blood pressure corresponding to each pulse duration (e.g., Pt in FIG. 1) of the PPG signal is then calculated using a blood pressure estimation model, and then a respiration rate is calculated according to a variation of a plurality of obtained blood pressures.

As mentioned above, a complete PPG waveform includes a mixed effect of systole and the pressure from blood vessel walls. In the present disclosure, a volume variation of blood vessels is detected by optical measurements to obtain said PPG signals.

As mentioned above, it is possible to use a PPG signal to indicate a frequency of heart circulation. As the PPG signal is obtained by optically detecting a volume variation of blood vessels and all blood vessels in the human body are connected together, related information of the blood pressure and the respiration cycle are obtainable from analyzed PPG signals.

For example, when a breathe-in occurs, muscular exertion squeezes blood vessels and causes a value of the PPG signal to rise up and a shape of the PPG signal to change; on the contrary, when a breathe-out occurs, muscle relaxation causes the value of the PPG signal to fall down and the shape of the PPG signal to change. Accordingly, it is able to identify the blood pressure and the respiration rate of a user by analyzing feature points of a PPG signal.

Furthermore, by comparing with the user's activity, it is possible to arrange a blood pressure detection system to output a prompt to indicate the blood pressure and the breathing state of a user. For example, when a user's blood pressure increases due to nervousness, it is able to suggest the user to relax using equipment which is coupled to the detected PPG signal; or when a user's blood pressure increases or decreases due to the weather change, it is able to suggest the user to change clothing. It is able to suggest the user by an auditory prompt such as a voice or music through a user's earphone, by a visual prompt through a user's portable device, or by the body sensing, e.g., the vibration. In addition, it is also possible to continuously record the variation of blood pressures during sleep to be served as data for long term monitoring of health.

One embodiment of obtaining the blood pressure and the respiration cycle Pb from a PPG signal is illustrated hereinafter.

Firstly, a PPG signal as shown in FIG. 1 is obtained by a blood pressure detection device, wherein the PPG signal 11 includes a plurality of feature points P_{S1}-P_{S4}, P_{S1}′-P_{S4}′. Next, it is able to obtain at least on blood pressure, as shown in FIG. 2, corresponding to each pulse duration Pt by recognizing a time difference between two feature points of the PPG signal 11 and using an estimation model (described below). FIG. 2 shows a continuous blood pressure signal formed by connecting a plurality of blood pressures with line segments, wherein the numeral 21 is referred to a systolic blood pressure signal and the numeral 22 is referred to a diastolic blood pressure signal.

In one embodiment of the present disclosure, the blood pressure detection device is further able to identify a rising part and a falling part of the blood pressure signals 21 and 22. As shown in one embodiment of FIG. 2, the rising part represents a breathe-in and the falling part represents a breathe-out. In other embodiments, corresponding to different estimation models, it is possible that the rising part represents a breathe-out and the falling part represents a breathe-in without particular limitations. After the above information is obtained, it is able to further calculate a respiration rate according to the blood pressure signals 21, 22 and to real-timely output at least one of the blood pressure signals and the respiration rate. In addition, it is possible to generate a prompt for the user's reference from a prompting device according to a comparison of comparing the blood pressure and/or respiration cycle with at least one threshold.

Therefore, by using the blood pressure detection device in the embodiment of the present disclosure, it is able to help a user to understand his/her physiological states more and achieve the effect of self-adjustment.

The present disclosure is also able to record user's blood pressures and breathing states for a long period of time to provide statistical data to the user as a reference for the self-adjustment, and it is possible to further determine thresholds according to said statistical data.

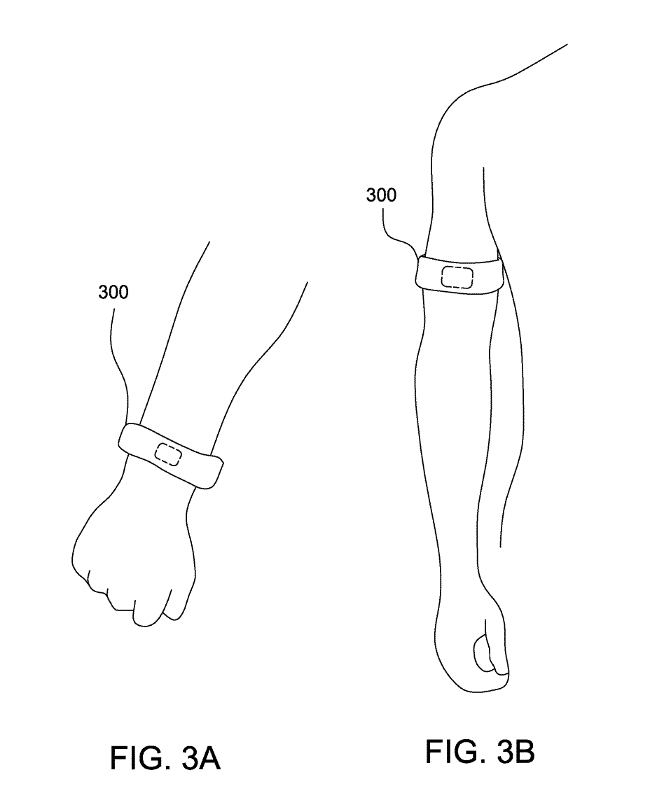

Please referring to FIGS. 3A and 3B, they are usage states of a blood pressure detection device according to some embodiments of the present disclosure. The blood pressure detection device 300 analyzes and displays the variation of a user's blood pressure signal changed with time by detecting a PPG signal of the user's skin tissues. Accordingly, the blood pressure detection device 300 is able to be arranged at any suitable location for detecting the PPG signal, e.g., setting on the user's wrist (FIG. 3A) or the user's arm (FIG. 3B), but not limited thereto. In another embodiment, the blood pressure detection device 300 is integrated in a portable electronic device or a wearable electronic device, e.g., a bracelet, an armband, a ring, a foot ring, a foot bracelet, a cell phone, an earphone, a headphone and a personal digital assistant (PDA) which contacts at least a part of skin surface of a user. In addition, the blood pressure detection device 300 is able to be coupled to a medical device, a home appliance, a vehicle, a security system in a wired or wireless way. Preferably, the one connected with the blood pressure detection device 300 includes a display device to real-timely display a detection result of the blood pressure detection device 300.

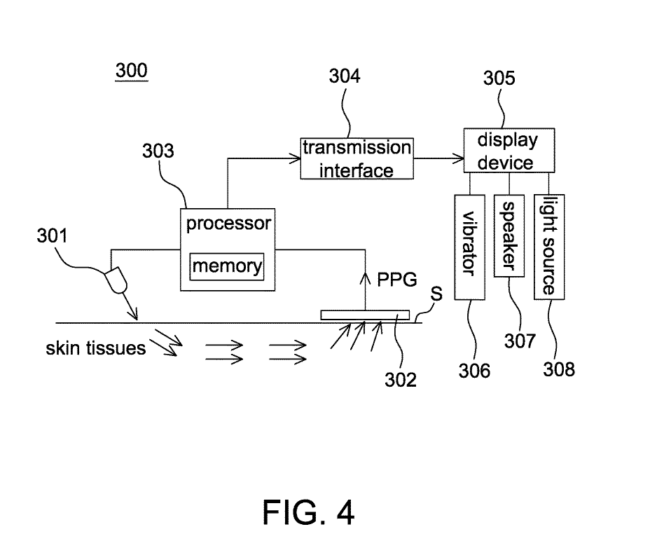

Please referring to FIG. 4, it is a schematic block diagram of a blood pressure detection device 300 according to one embodiment of the present disclosure. The blood pressure detection device 300 includes a light source 301, a light sensor 302 and a processor 303. In some embodiments, the blood pressure detection device 300 further includes a display device 305 configured to display a detection result of the blood pressure detection device 300. In some embodiments, the blood pressure detection device 300 further includes a transmission interface 304 coupled to an external display device 305 in a wired or wireless manner to output the detection result of the blood pressure detection device 300 to the display device 305 to be real-timely displayed. In other words, the display device 305 may or may not be included in the blood pressure detection device 300 depending on different applications.

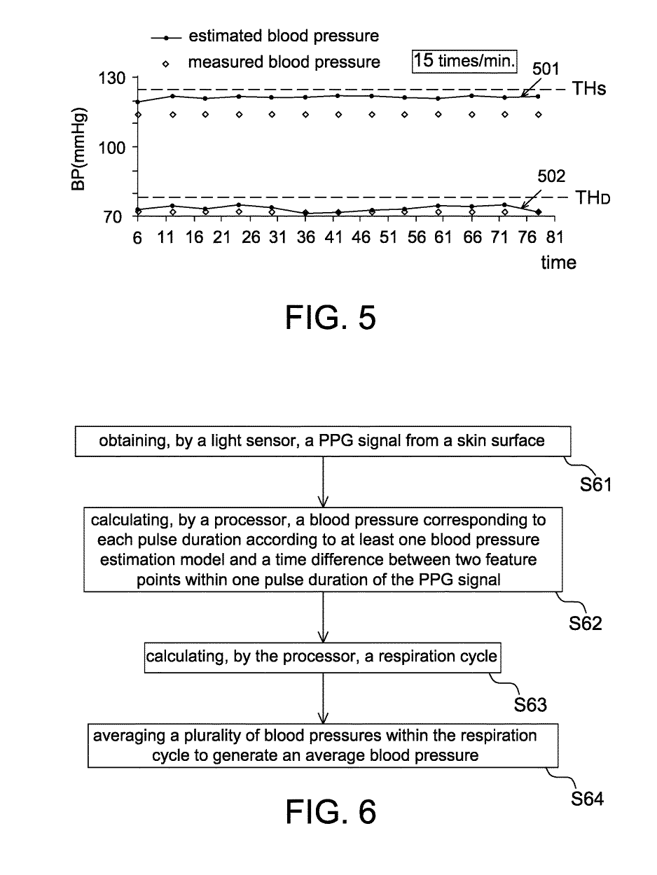

The display device 305 is, for example, a liquid-crystal display (LCD), a plasma display panel (PDP), an organic light-emitting diode (OLED) display or a projector for displaying images without particular limitations as long as it is able to display average blood pressures 501 and 502 (described later) as shown in FIG. 5 on a screen.

The light source 301 is, for example, a light emitting diode or a laser diode, and configured to emit light adapted to penetrate and be absorbed by skin tissues. For example, a wavelength of light emitted by the light source 301 is about 610 nm or 910 nm, but not limited thereto. The light source 301 illuminates a skin surface S to allow light to pass through skin tissues under the skin surface S. Preferably, the blood pressure detection device 300 includes a transparent surface to be attached to the skin surface S in operation and for protecting the light source 301, and the light source 301 is arranged at an inner side of the transparent surface. The transparent surface is made of transparent materials, e.g., plastic or glass, without particular limitations. In some embodiments, the transparent surface is a surface of a light guide which has the function of guiding light paths.

In some embodiments, when the blood pressure detection device 300 is also used to detect the blood oxygenation, the blood pressure detection device 300 includes two light sources to respectively emit light of different wavelengths, wherein a method of detecting the blood oxygenation may be referred to U.S. application Ser. No. 13/614,999 assigned to the same assignee of the present application, and the full disclosure of which is incorporated herein by reference.

The light sensor 302 is, for example, a photodiode or an image sensor array, e.g., a CMOS sensor array, and configured to detect ejected light emitted from the skin tissues to generate a PPG signal, as shown in FIG. 1 for example. The method of detecting and outputting a PPG signal by a photodiode is known to the art and thus details thereof are not described herein. The present disclosure is to identify the blood pressure and the respiration rate according to the detected PPG signal. A method of detecting a three dimensional physiology distribution by an image sensor array may be referred to U.S. application Ser. No. 14/955,463 assigned to the same assignee of the present application, and the full disclosure of which is incorporated herein by reference. Each pixel of the image sensor array respectively outputs the PPG signal mentioned herein, or an intensity sum of all pixels of the image sensor array is used as the PPG signal mentioned herein. Similarly, the light sensor 302 is arranged inside of the transparent surface.

The processor 303 is, for example, a microcontroller (MCU), a central processing unit (CPU) or an application specific integrated circuit (ASIC), which is electrically coupled to the light source 301 and the light sensor 302, and is configured to control the light source 301 and the light sensor 302 to operate correspondingly. The processor 303 calculates a blood pressure corresponding to each pulse duration Pt according to at least one blood pressure estimation model as well as a time difference between two feature points in one pulse duration Pt of the PPG signal 11, calculates a respiration cycle Pb, and averages a plurality of blood pressures within the respiration cycle Pb to generate an average blood pressure. In this embodiment, the average blood pressure is, for example, a relative value with respect to a real blood pressure of a user. The user is able to understand his/her blood pressure change according to the average blood pressure.

In one embodiment, the at least one blood pressure estimation model includes a polynomial taking the time difference between two feature points within one pulse duration Pt as a variable, wherein the polynomial is a linear polynomial, a quadratic polynomial or a higher order polynomial obtained by fitting a curve between the two feature points within the pulse duration Pt by a fitting method. The blood pressure estimation model is implemented by software and/or hardware, and integrated in the processor 303.

For example in FIG. 1, a plurality of feature points P_{S1}-P_{S4} are included within one pulse duration Pt, and said two feature points are selected from two of a maximum value P_{S2}, a second maximum value P_{S4}, a minimum value P_{S1} and a second minimum value P_{S3} within the pulse duration Pt of the PPG signal 11. In addition, the at least one blood pressure estimation model includes an estimation model for systolic pressure (SBP) and an estimation model for diastolic pressure (DBP).

The fitting method is used to fit the curve, for example, between the feature points P_{S1} and P_{S2}, between the feature points P_{S2} and P_{S3}, between the feature points P_{S3} and P_{S4}, between the feature points P_{S2} and P_{S1}′ or between the feature points P_{S2} and P_{S4} without particular limitations.

For example, one estimation model for systolic pressure is an equation (1) obtained according to the curve between the two feature points P_{S2} and P_{S1}′ (P_{S1}′ being the minimum value within a next pulse duration Pt), wherein a time difference between the feature points P_{S2} and P_{S1}′ is indicated as DT.

SBP=−0.095×DT+188.581 (1)

One estimation model for diastolic pressure is an equation (2) obtained according to the curve between the two feature points P_{S2} and P_{S4}, wherein a time difference between the feature points P_{S2} and P_{S4} is indicated as T1. To clearly show the time difference, T1 is shown by a time difference between the feature points P_{S2}′ and P_{S4}′ in FIG. 1, i.e., summing time differences between feature points P_{S2}′ and P_{S4}′ and between feature points P_{S2} and P_{S4} being identical.

DBP=−0.344×T₁+174.308 (2)

The above diastolic blood pressure model and systolic blood pressure model are only intended to illustrate and able to obtain rough values of the blood pressure corresponding to the measured PPG signal. If a more precise model is required, it is possible to use other mathematical models without particular limitations, e.g., using a higher order polynomial having more variables or more different time differences.

For example, it is possible to represent the SBP and DPP by an equation ΣaₖXₖ^{K}+C, wherein Xₖ indicates a time difference and C is a known constant.

One estimation model for systolic pressure and one estimation model for diastolic pressure are obtainable between arbitrary two feature points, and only values of the estimated blood pressure are different. In this embodiment, the estimation model for systolic pressure and the estimation model for diastolic pressure are preferably a common model obtained by gathering statistics of PPG signals of some people before shipment and stored in a nonvolatile memory of the processor 303. It should be mentioned that said feature points are not limited to those given in the present disclosure and may be determined by positions in the PPG signal that correspond to maximum/minimum values in a linear differential curve or a quadratic differential curve of the PPG signal. In addition, a number of feature points for determining the blood pressure estimation model is not limited to 2.

The processor 303 obtains one systolic blood pressure respectively corresponding to each pulse duration Pt, for example, using the equation (1) to form a systolic blood pressure signal 21 as shown in FIG. 2, wherein each dot in the systolic blood pressure signal 21 is one systolic blood pressure obtained by the equation (1), and these dots are connected by line segments. The processor 303 obtains one diastolic blood pressure respectively corresponding to each pulse duration Pt, for example, using the equation (2) to form a diastolic blood pressure signal 22 as shown in FIG. 2, wherein each dot in the diastolic blood pressure signal 22 is one diastolic blood pressure obtained by the equation (2), and these dots are also connected by line segments.

In this embodiment, the processor 303 further calculates a respiration cycle Pb according to the systolic blood pressure signal 21 and/or the diastolic blood pressure signal 22. In one embodiment, the processor 303 uses a fast Fourier transform (FFT) to convert a plurality of blood pressures of the systolic blood pressure signal 21 and/or the diastolic blood pressure signal 22 to a frequency domain and then obtains the respiration cycle Pb. In another embodiment, the processor 303 calculates the respiration cycle Pb using a time difference between two adjacent minimum blood pressures among a plurality of blood pressures of the systolic blood pressure signal 21 and/or the diastolic blood pressure signal 22. In other words, it is possible to take a period of the systolic blood pressure signal 21 and/or the diastolic blood pressure signal 22 as the respiration cycle Pb.

It is seen from FIG. 2 that the systolic blood pressure signal 21 and the diastolic blood pressure signal 22 obtained by the processor 303 change with the breathing of a user and are unstable in amplitude. Accordingly, the processor 303 further calculates an average value (e.g., the root-mean-square, but not limited to) of a plurality of blood pressures within the respiration cycle Pb so as to obtain the average blood pressure as shown in FIG. 5, wherein the numeral 501 is referred to the systolic blood pressure and the numeral 502 is referred to the diastolic blood pressure. FIG. 5 is obtained by setting the respiration cycle Pb as 6 seconds. The interval of the respiration cycle Pb is different according to different scenarios. It can been seen from FIG. 5 that the average blood pressure shown in FIG. 5 is much more stable than the blood pressure in FIG. 2.

Referring to FIG. 5 again, it further shows a plurality of measured blood pressures measured by a hemadynamometer. As mentioned above, the blood pressure detection device 300 of the present disclosure measures relative blood pressures. In some embodiments, the memory further stores at least one calibration value, wherein the calibration value is a difference value between a measured blood pressure measured by a hemadynamometer and an estimated blood pressure calculated by the blood pressure estimation model of the present disclosure. Accordingly, after the processor 303 obtains the estimated blood pressure (e.g., the average blood pressure 501 and 502), the calibration value is added to or subtracted from the estimated blood pressure for calibration thereby obtaining a more accurate individualized blood pressure, i.e. calibration values are measured and stored corresponding to different users respectively so as to obtain individualized calibration values.

The transmission interface 304 outputs at least one of the average blood pressure and a respiration rate in a wired or wireless way, e.g., outputting data of at least one of the average blood pressure and a respiration rate at a predetermined frequency to a display device 305 for real-time display, wherein said wired and wireless transmission techniques are known to the art and thus details thereof are not described herein. The respiration rate is obtainable according to the respiration cycle Pb, e.g., a reciprocal of the respiration cycle Pb multiplied by 60 seconds. It is appreciated that when the blood pressure detection device 300 also includes the display device 305, the transmission interface 304 is not implemented, or the transmission interface 304 is arranged inside the blood pressure detection device 300 between the processor 303 and the display device 305.

The display device 305 real-timely displays a variation curve of the average blood pressure (e.g., the estimated blood pressures 501 and 502 shown in FIG. 5) changed with time and/or values of the respiration rate. In addition, the processor 303 further reads at least one blood pressure threshold THs, TH_{D} associated with the blood pressure from the memory, and sends the read values to the display device 305 directly or via the transmission interface 304 to be displayed thereon. For example, lines, numbers or graphics are shown on a screen of the display device 305 to mark the blood pressure thresholds THs, TH_{D} and values of the respiration rate to allow a user to easily observe his/her blood pressures and breathing states from the display device 305.

Different from conventional blood pressure detection devices, the blood pressure detection device 300 of the present disclosure is able to real-timely display the blood pressure and breathing state of a user. In other words, as the blood pressure detection device 300 analyzes a PPG signal detected by the light sensor 302, when the processor 303 receives the PPG signal, the processor 303 starts to analyze and output the blood pressure signals 21 and 22 and/or the respiration rate to the display device 305 to be displayed thereon. As the processor 303 averages the blood pressure signals 21 and 22 by the respiration cycle Pb, the display device 305 is able to display blood pressures after one respiration cycle Pb. Generally, under normal condition, the respiration cycle Pb is about 5 to 6 seconds. It is appreciated that different users have different respiration cycles Pb, and different scenarios cause different respiration cycles Pb. In other embodiments, when the processor 303 does not calculate the average blood pressure, the display device 305 real-timely displays, for example, blood pressure signals 21 and 22 as shown in FIG. 2.

In addition, to improve the user experience, the blood pressure detection device 300 further includes a prompt device to output a prompt signal according to a comparison of comparing at least one threshold with the average blood pressure and/or respiration cycle, wherein the prompt signal is, e.g., a vibration signal, a light signal, an audio signal and/or an image signal without particular limitations as long as the user can be informed.

The blood pressure detection device 300 of the present disclosure is applicable to adjusting the emotion as well as the work and rest.

For example, when a user's average blood pressure does not reach or exceeds the blood pressure thresholds TH_{S} and TH_{D}, the prompt device 305 outputs a prompt signal. Accordingly, the user changes emotion, takes medicine, puts on or takes off clothes and so on to allow the average pressure to return to a normal status.

For example, when a user's respiration rate does not reach or exceeds a threshold, the prompt device 305 outputs a prompt signal. A frequency value of the respiration rate and the estimated blood pressures 501 and 502 are shown together on a screen. As mentioned above, the processor 303 is able to obtain the respiration rate within one respiration cycle Pb without accumulating count values for a whole minute.

The indicating method of the prompt signal is determined according to different applications.

For example, the display device 305 may also be used as the prompt device. When the average blood pressure and/or the respiration rate exceed or do not reach the threshold, the processor 303 provides image signals to the display device 305 to make the display device 305 display the prompt, e.g., by words, graphs, brightness and so forth.

For example, the blood pressure detection device 300 further includes a vibrator 306 used as the prompt device. When the average blood pressure and/or the respiration rate exceed or do not reach the threshold, the processor 303 provides vibration signals to the vibrator 306 to make the vibrator 306 generate vibrations to warn the user.

For example, the blood pressure detection device 300 further includes a speaker 307 used as the prompt device. When the average blood pressure and/or the respiration rate exceed or do not reach the threshold, the processor 303 provides voice signals to the speaker 307 to make the speaker 307 generate sounds to warn the user.

For example, the blood pressure detection device 300 further includes a warning light source 308 used as the prompt device. When the average blood pressure and/or the respiration rate exceed or do not reach the threshold, the processor 303 provides optical signals to the warning light source 308 to make the warning light source 308 illuminate light to warn the user.

In some embodiments, the processor 303 is built-in, for example, a learning algorithm (e.g., implemented by software and/or hardware), to determine the above thresholds, e.g., blood pressure threshold and the respiration rate threshold, but not limited thereto, according to the user's historical records. For example, the thresholds are divided into sleep time, work time, sports time and so forth. Information related to the historical records is stored in, for example, a non-volatile memory.

Please referring to FIG. 6, it is a flow chart of an operating method of a blood pressure detection device according to one embodiment of the present disclosure, which includes the steps of: obtaining, by a light sensor, a PPG signal from a skin surface (step S61); calculating, by a processor, a blood pressure corresponding to each pulse duration according to at least one blood pressure estimation model and a time difference between two feature points within one pulse duration of the PPG signal (step S62); calculating, by the processor, a respiration cycle (step S63), and averaging, by the processor, a plurality of blood pressures within the respiration cycle to generate an average blood pressure (step S64).

Step S61: The blood pressure detection device 300 is preferably fixed with respect to a skin surface S in operation such that a PPG signal detected by the light sensor 302 is not affected by noises due to movement. In addition, the processor 303 is further built-in with an algorithm for eliminating the noises in PPG signals caused by the movement, wherein a method of eliminating motion noises may be referred to U.S. application Ser. No. 13/614,999 assigned to the same assignee of the present application, and the full disclosure of which is incorporated herein by reference.

Step S62: The processor 303 starts to identify feature points within one pulse duration Pt (e.g., P_{S1}-P_{S4} in FIG. 1) of a PPG signal right after receiving the

PPG signal from the light sensor 302, wherein said identifying is implemented by software and/or hardware. At least one blood pressure estimation model is pre-stored in a memory, wherein the at least one blood pressure estimation model includes a polynomial using a time difference between two feature points within one pulse duration Pt as a variable, e.g., equations (1) and (2). The processor 303 puts the time differences (e.g., ST, DT, T1) between the measured feature points P_{S1}-P_{S4} into the at least one blood pressure estimation model to calculate a blood pressure corresponding to each pulse duration Pt, as shown in FIG. 2.

Steps S63-S64: After obtaining a plurality of blood pressures, the processor 303 calculates a respiration cycle Pb (as shown in FIG. 2) directly in the time-domain or calculates the respiration cycle Pb in the frequency domain by using the fast Fourier transform (FFT). In this embodiment, the respiration cycle Pb is for calculating a respiration rate to be displayed by the display device 305 and for averaging a plurality of blood pressures calculated by the processor 303 to obtain the estimated blood pressures 501 and 502 as shown in FIG. 5.

Next, the respiration rate and/or the estimated blood pressures 501 and 502 are sent to a display device 305 to be real-timely displayed thereon. In addition, the processor 303 further compares the respiration rate and/or the estimated blood pressures 501, 502 with at least one threshold to confirm whether values thereof are within a normal range to accordingly generate a prompt signal.

In some embodiments, to obtain personalized blood pressures, a difference value between an estimated blood pressure and a measured blood pressure of a hemadynamometer is stored in a memory to be used as a calibration value, wherein the calibration value is stored by an application (APP) in a calibration stage, e.g., a user inputting the difference value between the estimated blood pressures and measured blood pressures in FIG. 5 into a user interface to be stored. During operation, the processor 303 automatically calibrates the estimated blood pressures 501 and 502 with the stored calibration value.

It should be mentioned that, it is possible that the display device 305 displays the estimated blood pressures 501, 502 and the respiration rate but does not display the measured blood pressures. In some embodiments, the display device 305 further displays the systolic blood pressure signal 21 and/or the diastolic blood pressure signal 22 depending on applications thereof.

It should be mentioned that although the above embodiments take the reflective optical blood pressure detection device as an example, it is only intended to illustrate but not to limit the present disclosure. In other embodiments, the blood pressure detection device is a transmissive optical device in which disposed positions of the light source and the light sensor are different from the above embodiments but the sensing theory is not changed, and thus details thereof are not repeated herein.

It should be mentioned that in the above embodiments a memory disposed in the processor 303 is taken as an example for illustration purposes, but the present disclosure is not limited thereto. In other embodiments, the memory 303 is located outside of the processor 303 without particular limitations as long as the processor 303 is able to access the memory.

In addition, in some embodiments, when the processor 303 identifies that the variation of obtained blood pressures (e.g., the standard deviation) exceeds a predetermined range, the calculation of the average blood pressure or the outputting of estimated blood pressures being obtained is stopped till the obtained blood pressures return to the predetermined range.

As mentioned above, conventional blood pressure detection devices are not able to real-timely display the user's blood pressures and to perform the long term monitoring such that applications thereof are limited. Therefore, the present disclosure further provides a blood pressure detection device (as shown in FIG. 4) and an operating method thereof (as shown in FIG. 6) that real-timely calculate and display blood pressures and breathing states of a user. In addition, the blood pressure detection device of the present disclosure is further able to help a user to adjust his/her physiology states by a prompting mechanism to effectively enhance the user experience and applicable ranges.

Although the disclosure has been explained in relation to its preferred embodiment, it is not used to limit the disclosure. It is to be understood that many other possible modifications and variations can be made by those skilled in the art without departing from the spirit and scope of the disclosure as hereinafter claimed.

What is claimed is:

1. A blood pressure detection device comprising:
a light source configured to illuminate a skin surface to allow light to pass through skin tissues under the skin surface;
a light sensor configured to detect ejected light from the skin tissues to generate a photoplethysmography (PPG) signal; and
a processor configured to
calculate at least one blood pressure corresponding to each pulse duration according to at least one blood pressure estimation model and a time difference between two feature points within one pulse duration of the PPG signal, wherein the at least one blood pressure estimation model comprises a polynomial using the time difference between the two feature points within one pulse duration as a variable,
calculate a respiration cycle, and
average a plurality of blood pressures within the respiration cycle to generate an average blood pressure.

2. The blood pressure detection device as claimed in claim 1, wherein the two feature points are two of a maximum value, a second maximum value, a minimum value and a second minimum value within the pulse duration of the PPG signal.

3. The blood pressure detection device as claimed in claim 1, wherein the processor is configured to calculate the respiration cycle according to a plurality of blood pressures using a fast Fourier transform.

4. The blood pressure detection device as claimed in claim 1, wherein the processor is configured to calculate the respiration cycle using a time difference between two adjacent minimum blood pressures among a plurality of blood pressures.

5. The blood pressure detection device as claimed in claim 1, wherein the at least one blood pressure estimation model comprises an estimation model for systolic pressure and an estimation model for diastolic pressure.

6. The blood pressure detection device as claimed in claim 1, further comprising a display device configured to real-timely display at least one of the average blood pressure and a respiration rate, wherein the respiration rate is obtained according to the respiration cycle.

7. The blood pressure detection device as claimed in claim 1, further comprising a prompt device configured to generate a prompt signal according to at least one of the average blood pressure and the respiration cycle.

8. The blood pressure detection device as claimed in claim 1, wherein the light sensor is a photodiode or an image sensor array.

9. The blood pressure detection device as claimed in claim 1, wherein the blood pressure detection device is integrated with a portable electronic device or a wearable electronic device.

10. A blood pressure detection device comprising:
a light source configured to illuminate a skin surface to allow light to pass through skin tissues under the skin surface;
a light sensor configured to detect ejected light from the skin tissues to generate a photoplethysmography (PPG) signal;
a memory configured to store at least one calibration value, wherein the calibration value is a difference value between a measured blood pressure of a hemadynamometer and an estimated blood pressure; and
a processor configured to
calculate a blood pressure corresponding to each pulse duration according to at least one blood pressure estimation model and a time difference between two feature points within one pulse duration of the PPG signal, wherein the at least one blood pressure estimation model comprises a polynomial using the time difference between the two feature points within one pulse duration as a variable,
calculate a respiration cycle,
average a plurality of blood pressures within the respiration cycle to generate an average blood pressure, and
calibrate the average blood pressure with the calibration value.

11. The blood pressure detection device as claimed in claim 10, wherein the two feature points are two of a maximum value, a second maximum value, a minimum value and a second minimum value within the pulse duration of the PPG signal.

12. The blood pressure detection device as claimed in claim 10, wherein the processor is configured to calculate the respiration cycle according to a plurality of blood pressures using a fast Fourier transform.

13. The blood pressure detection device as claimed in claim 10, wherein the processor is configured to calculate the respiration cycle using a time difference between two adjacent minimum blood pressures among a plurality of blood pressures.

14. The blood pressure detection device as claimed in claim 10, wherein the at least one blood pressure estimation model comprises an estimation model for systolic pressure and an estimation model for diastolic pressure.

15. The blood pressure detection device as claimed in claim 10, further comprising a transmission interface configured to output at least one of the average blood pressure and a respiration rate to a display device, wherein the respiration rate is obtained according to the respiration cycle.

16. The blood pressure detection device as claimed in claim 10, wherein the light sensor is a photodiode or an image sensor array.

17. An operating method of a blood pressure detection device, the blood pressure detection device comprising a light sensor and a processor, the operating method comprising:
obtaining, by the light sensor, a photoplethysmography (PPG) signal from a skin surface;
calculating, by the processor, a blood pressure corresponding to each pulse duration according to at least one blood pressure estimation model and a time difference between two feature points within one pulse duration of the PPG signal, wherein the at least one blood pressure estimation model comprises a polynomial using the time difference between the two feature points within one pulse duration as a variable;
calculating, by the processor, a respiration cycle; and
averaging, by the processor, a plurality of blood pressures within the respiration cycle to generate an average blood pressure.

18. The operating method as claimed in claim 17, further comprising:
calibrating, by the processor, the average blood pressure with a calibration value, wherein the calibration value is a difference value between a measured blood pressure of a hemadynamometer and an estimated blood pressure.

19. The operating method as claimed in claim 17, wherein the two feature points are two of a maximum value, a second maximum value, a minimum value and a second minimum value within the pulse duration of the PPG signal.

20. The operating method as claimed in claim 17, wherein the at least one blood pressure estimation model comprises an estimation model for systolic pressure and an estimation model for diastolic pressure.
